(19) United States
(12) Patent Application Publication
(10) Pub. No.: US 2010/0016813 A1
(43) Pub. Date: Jan. 21, 2010

(54) PRODUCT FOR TREATING HEEL FISSURES

(75) Inventors: IVAN E. BROWN, Spirit Lake, IA (US); RONALD S. KRIVOSHA, Bellevue, WA (US)

Correspondence Address: MCKEE, VOORHEES & SEASE, P.L.C., 801 GRAND AVENUE, SUITE 3200, DES MOINES, IA, 50309-2721, US

(73) Assignee: BROWN MEDICAL INDUSTRIES, Spirit Lake, IA (US)

(21) Appl. No.: 12/175,646

(22) Filed: Jul. 18, 2008

(51) Int. Cl. A61M 35/00

(52) U.S. Cl. 604/293

(57) ABSTRACT

A device for treating heel fissures (cracked heels) comprised of an elastic sleeve adapted to fit over the foot and heel of a person with toes and the back of the heel exposed, with the sleeve defining an exterior surface and an interior surface. Attached to the interior surface is at least one exfoliating insert, located to rub against callus areas of the foot as a person moves about during sleep.

### FIELD OF THE INVENTION

The present invention relates generally to a device treating heel fissures (cracked heels).

### BACKGROUND OF THE INVENTION

Heel fissures form at the intersection of two types of skin in the foot, glabrous and hairy skin, that come together at the side and bottom of the heel. For most people, the heel rim is a smooth transition between these two types of skin. However, when there is an uneven transition between the two skin types, calluses and heel fissures can occur. These fissures are secondary to recurrent callus formation. As the callus surrounding the rim of the heel thickens, the callus cracks. There is no cure, but prevention of deep, infected fissures is important, and that prevention requires a combination of exfoliation, moisturizing, and avoidance of the environmental conditions that seem to exacerbate the condition.

A combination of professional and personal care are used to help someone with heel fissures prevent infection. Regular pedicures by a nail technician, combined with daily exfoliation and moisturizing cream at home can help. Additionally, minimizing the time spent standing, wearing open back shoes, and being cognizant of other environmental factors that cause callous formation will help minimize their effect.

While these described procedures can help, those suffering from callused heels often fail to engage in the proper professional and personal care on a sufficiently regular basis to alleviate the problem. Largely because it takes so much time from a person's daily routines.

Currently, home remedies fall into one or two categories: exfoliation or moisturizing. Beyond the traditional, inexpensive pumice stone, more sophisticated electronic sloughing devices such as the Artemis Woman Heel Smoother are currently available at about $20-$30, depending on the model. Moisturizing creams for cracked heels abound, and there are some socks/booties that claim to have moisturizing properties built in to help soften callouses.

Given the above state of the art, it can be seen that there is a need for something that passively meets the problem of heel calluses, cracks and fissures over a long period of time, without the worry, need and expense of a periodic professional personal care.

The current inventors and their common assignee Brown Medical currently sell stabilizing devices for heel pain that include elastic sleeves such as those described in U.S. Pat. No. 5,645,525 and U.S. Pat. No. 7,056,299. The present invention represents a variation of those devices that occurred to the inventors to specifically and inexpensively treat heel fissures.

Put another way, it is a primary objective of the present invention to provide an elastic sleeve medical device that can simply and passively treat heel fissures, for example during sleeping time.

The above primary objective is provided with a device which fits like a stocking and feels comfortable such that it can be easily worn to bed at night, remains cool during sleeping, and the natural thrashing about that occurs during sleep then exfoliates heel fissures.

The method of accomplishing this as well as other objectives of the invention such as avoidance of expensive mechanical devices, avoidance of the regular use of pumice stone, and avoidance of expensive moisturizers, will be apparent from the detailed description which follows hereinafter.

### BRIEF SUMMARY OF THE INVENTION

A device for treating heel fissures (cracked heels) comprised of an elastic sleeve adapted to fit over the foot and heel of a person with toes and the back of the heel exposed, with the sleeve defining an exterior surface and an interior surface. Attached to the interior surface is at least one exfoliating insert, located to rub against callus areas of the foot as a person moves about during sleep.

### BRIEF DESCRIPTION OF THE DRAWINGS

FIG. 1 is an isometric view showing one embodiment of the present invention.

FIG. 2 is a perspective view of the embodiment shown in FIG. 1, showing relative location of inserts for exfoliating the heel.

FIG. 3 is a top view of the embodiment shown in FIG. 2.

FIG. 4 is a rear view of a foot device of the present invention.

### DETAILED DESCRIPTION OF THE PREFERRED EMBODIMENT

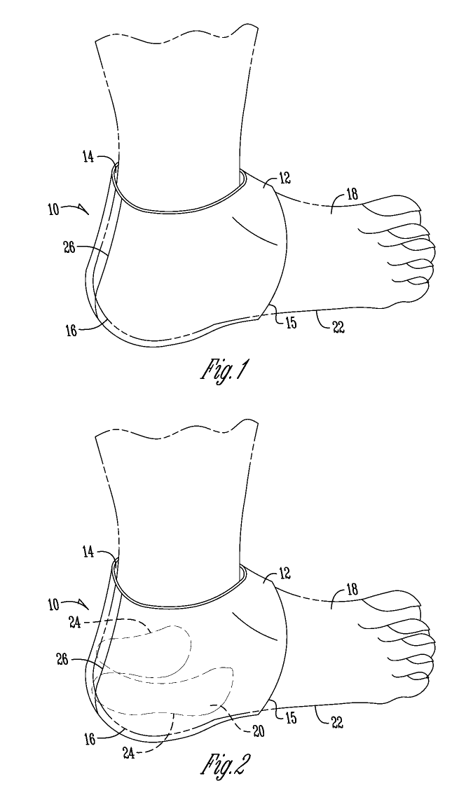

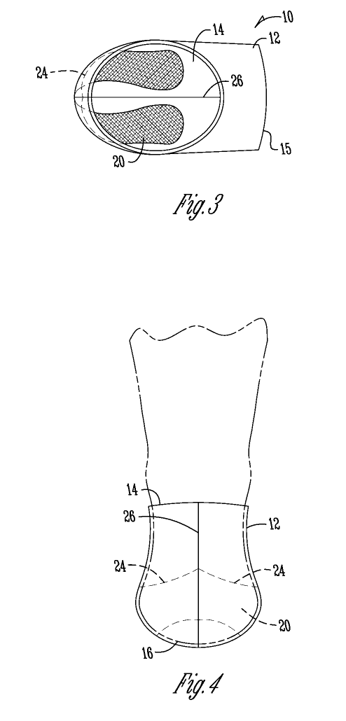

FIG. 1 shows the preferred embodiment of the present invention. The exfoliating device 10, is comprised of a half stocking 12 (or sleeve) having an open top 14 and an open forward end 15. The stocking is made of a stretchable material so as to fit snugly around the posterior, lateral, medial, and plantar aspects of the heel 16 of the foot 18. Device 10 includes insert exfoliating pads 20 and 22 on each side of the inside surface of the half stocking 12 proximate the calcaneus of the heel 16. As shown in FIG. 5, the pads 20 and 22 are disposed in a pocket formed by, for example, sewing a piece of pumice covered material 40 to the interior of sleeve 12. The stitching 24 is shown on the exterior surface of half stocking 12. When the half stocking 12 is positioned over the heel 16, it extends forward along the lateral and medial sides of the foot 18 to approximately the mid-point of the longitudinal arch 24 of the foot 18 (FIGS. 1 and 2). It is important to note that the open top 14 of the half stocking 12 terminates at the calcaneus below the talo calcaneal joint of the foot 18. Thus the foot forward portion and the heel can remain cool and comfortable during sleep.

Device 10 has mirror image left and right lateral portions, such that the device can be worn on either foot 18. The device 10 can also be used alone or in conjunction with any type of foot wear. Device 10 is also stretchable or elastic so that it may be used with different foot sizes.

Stocking 12 is preferably comprised of an elastic firm stitched material forming an elastic sleeve and is adapted to slide over the foot 18 of the user as shown in the figures to provide a tight fit around the heel 16. An example of acceptable material is neoprene. Preferably the material is cut and stitched so that the seam 26 extends centrally along the bottom of the foot and up the back of the heel.

While the device is primarily for treating heel fissures, it may also be used to stabilize the calcaneus and reduce movement and irritation by applying pressure on opposite sides of the ankle. It should be understood however that the primary purpose of this device is not ankle support but is for treating heel fissures.

Pads 20 and 22 are preferably comprised of a pair of exfoliating insert pads sewn into the interior of the sleeve 12.

On its skin exposed side the insert pads 20 and 22 have an attached pumice surface 22. Pads 20 and 22 are spaced apart and disposed on opposite sides of the inside surface of the elastic sleeve 12 such that when the device is worn around the heel 16, as the wearer's foot thrashes and moves to and fro during a night's sleep, the pads rub against calluses and heel fissures, exfoliating them. In the morning the pad is removed and again put on at night. In this way, the patient does not have to worry and exfoliation passively occurs during sleep.

In the preferred embodiment pads 20 and 22 are shaped as shown in FIGS. 1 and 2. They are designed to compress against calluses of the heel exerting pressure due to the elastomeric nature of sleeve 12. Pads 20 and 22 may take on many shapes, the elongated embodiment shown in the figures only being one example.

Pads 20 and 22 exert equal and opposite compressive forces against the skin as the device 10 is worn. Pads 20 and 22 are disposed at sufficiently low positions against the user's foot that they rub against the heel transition point between the two types of skin, glabrous and hairy skin. Device 10 is normally used passively during sleeping. It may also be used with a shoe sock or slipper if one desires.

Other optional changes may also be made. For example, this application has been described particularly with respect to a pumice covered insert but other abrasive materials may be used, such as abrasive silicon dioxide, covered inserts as used on nail files for example.

The point being that when the sleeve 12 is worn, fissures caused by a number of factors including predisposition to dry skin, living in a dry climate, being overweight or obese, prolonged standing, wearing open back shoes and other skin conditions such as psoriasis and edema which the general population frequently suffer from, can be passively treated. The exfoliation provided by the device allows the user to treat cracked heels without actively scrubbing with a pumice stone or other device, thus allowing them to treat the problem over a longer period of time with less effort and more comfort and less expense.

The preferred embodiment of the present invention has been set forth in the drawings and specification, and although specific terms are employed, these are used in a generic or descriptive sense only and are not used for purposes of limitation. Changes in the form and proportion of parts as well as in the substitution of equivalents are contemplated as circumstances may suggest or render expedient without departing from the spirit and scope of the invention as further defined in the following claims.

What is claimed is:

1. A device for treating heel fissures, comprising:
an elastic sleeve adapted to be fit around the foot and the heel of a person, the sleeve having a front toe opening and a top ankle opening and having an exterior surface and an interior surface; and
at least one exfoliating insert attached to the interior surface of the elastic sleeve at a location to allow the wearer to passively exfoliate callused skin.

2. The device of claim 1 wherein they are at least two exfoliating insert pads.

3. The device of claim 2 wherein the exfoliating insert pads have an exposed surface covered with pumice.

4. The device of claim 1 wherein the at least one exfoliating insert has an exposed surface covered with a non-pumice abrasive material.

5. The device of claim 1 wherein the elastic sleeve has mirror image left and right portions so as to be interchangeably worn on either the left or the right foot of a person.

6. A device for exfoliating heel fissures, comprising:
an elastic sleeve adapted to be fit around the foot and heel of a person, the sleeve having a front toe opening and a top ankle opening and a back heel opening, and an exterior surface and an interior surface;
a pair of exfoliating pads positioned on opposite sides of the interior of the elastic sleeve located to allow the wearer to exfoliate callused skin passively during rest.

7. The device of claim 6 wherein there are more than a pair of exfoliating pads.

8. The device of claim 7 wherein the pads have a surface covered with pumice.

9. The device of claim 6 wherein the pads have an exterior surface covered with non-pumice abrasive material.

10. The device of claim 6 wherein the sleeve has mirror image left and right portions so as to be interchangeably worn on either the left or the right foot of a person.

11. The method for passively exfoliating heel fissures comprising the steps of:
placing an elastic sleeve having a front toe opening and a top ankle opening and a rear heel opening around the foot and heel of a person;
attaching at least one exfoliating insert pad to the interior surface of the elastic sleeve at a location to allow the wearer to exfoliate callused skin of the foot; and
putting said elastic sleeve on at least one foot and wearing it to bed to allow exfoliation to passively occur during sleep.
